(19) United States
(12) Patent Application Publication
(10) Pub. No.: US 2009/0227897 A1
(43) Pub. Date: Sep. 10, 2009

(54) PATIENT IDENTIFICATION FOR POINT OF CARE DIAGNOSTICS

(75) Inventors: Matthias Wendt, Wuerselen (DE); Andreas Boos, Bondorf (DE); Gerhard Spekowius, Roetgen (DE); Peter K. Bachmann, Wuerselen (DE); Hans Nikol, Aachen (DE); Helga Hummel, Aachen (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., Eindhoven (NL)

(21) Appl. No.: 11/720,875

(22) PCT Filed: Dec. 5, 2005
(86) PCT No.: PCT/IB2005/054057

§ 371 Date: Jun. 5, 2007

Related U.S. Application Data
(60) Provisional application No. 60/634,674, filed on Dec. 9, 2004.

(51) Int. Cl. A61B 5/15; C12Q 1/68; G06F 7/04

(52) U.S. Cl. 600/583; 600/573; 435/6; 340/5.82

(57) ABSTRACT

A patient presses a finger concurrently against a needle (14) of a collection container (12) and a fingerprint surface (22, 52). In one embodiment, the patient's fingerprint is read electronically by a scanner (24) and correlated with a container serial number (16). In another embodiment, the patient's fingerprint is embossed in a foil layer (52). An analyzer assembly (40, 40′ 40″) analyzes the biological sample and reads either the container serial number or scans the fingerprint carrying layer (52) and sends the test results and either the fingerprint or container identification to a patient memory (30). Using the fingerprint information, a processor (32) correlates the test results with a record (60) of a corresponding patient. Alternately or additionally, the analyzer assembly includes a DNA analyzer (70) which measures preselected DNA characteristics (76) of the sample. The processor (32) compares the measured DNA subset characteristics with DNA subset characteristics (66) in the patient records to correlate or confirm the correlation of the analysis results with the proper patient.

The present invention pertains to point of care diagnostics (POCD) and will be described with particular reference thereto. However, it is to be appreciated that the invention may also find application in other areas which involve the collection and analysis of blood, tissue, or other bodily materials.

Currently, blood and other tissue and fluid samples are taken from a patient and placed in an appropriate, usually sealed container. In some instances, the sample is analyzed with automated analysis equipment which is located in the same room as the patient. Once the sample is analyzed, a human operator correlates the test results with the specific patient. In other instances, the attendant marks the containers with an identification of the patient and the containers are sent to a central laboratory for analysis. Often, the containers are marked with a bar code which is read by the analysis equipment such that the test results are automatically correlated with the bar code identification. However, a human operation is still used to correlate a specific patient with the bar code.

Although the medical personnel are well-trained, these manual steps still raise the possibility for human error. Because a course of treatment is often predicated on the analysis results, any instances of human error can have dire consequences.

The present application is directed to reducing or eliminating the potential for human error.

In accordance with one aspect of the present invention, a point of care diagnostics system is provided. A container receives a biological sample from a patient. An analyzer assembly receives the biological container and generates both analysis results of the biological sample and a biometrically based patient indicator.

In accordance with another aspect of the present invention, a method of point of care diagnosing is provided. A biological sample is taken of a patient. The sample is analyzed and the analysis results are generated along with a biometrically based patient identification for use in correlating the analysis results with the patient from whom the biological sample was taken.

One advantage of the present invention resides in a reduction in the potential for human error.

Another advantage of this invention resides in an improved confidence that analytical results are attributed to the proper patient.

Yet another advantage of this invention resides in increased assurance that a patient is receiving a proper course of treatment.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

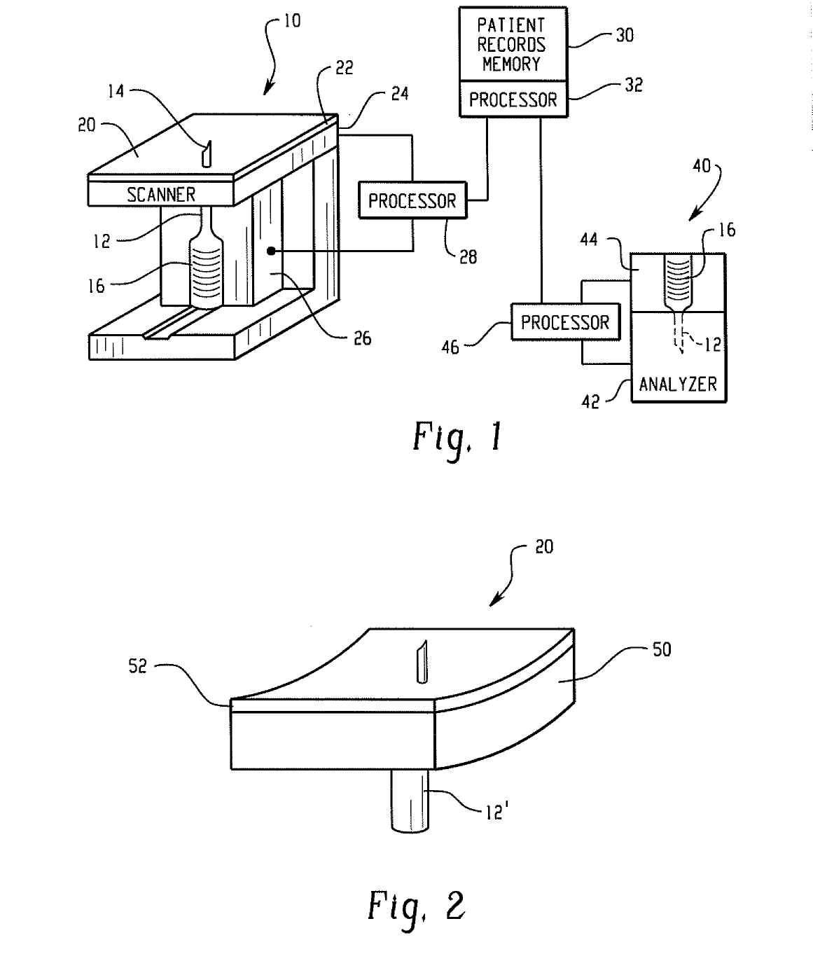

FIG. 1 is a diagrammatic illustration of a blood sampling and analysis system in accordance with the present invention;

FIG. 2 illustrates an alternate embodiment of a blood sampling system;

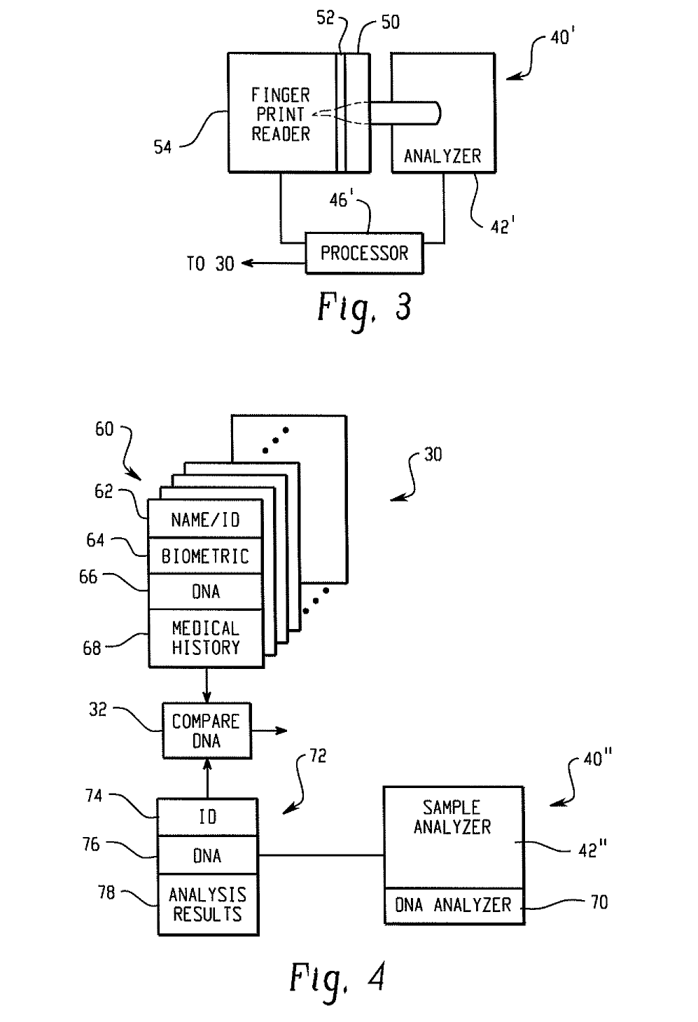

FIG. 3 is a diagrammatic illustration of an alternate embodiment of an analyzer system; and,

FIG. 4 illustrates another embodiment of the present invention.

With reference to FIG. 1, a body fluid or tissue sampling device 10 includes a container, such as a capillary tube 12, for receiving the blood or other tissue or fluid samples. The capillary tube includes a needle 14 for puncturing a patient's finger to get a small sample of blood which is drawn into the capillary tube. The capillary tube further includes a label 16 which carries a unique serial number in a machine-readable format.

The needle end 14 of the capillary tube projects through a biometric sensor surface 20. More specifically to the illustrated embodiment, the sensor surface 20 is a glass or transparent plate 22 below which a fingerprint scanner 24 is mounted. When the patient presses its finger against the glass plate 22 and the capillary needle 14 to draw the blood sample, the scanner reads the patient's fingerprint. Concurrently, a reader 26 reads the unique identification on the label 16. A processor 28 receives the fingerprint information from the scanner and the unique serial number from the reader 26 and provides this information to a central hospital patient records memory 30.

In an alternate embodiment, the label reader 26 is replaced with a label printer. The processor 28 communicates biometric information from the scanner 24 to the printer to print the biometric identification as a coded identification of the patient or to the central memory to determine the identity of the patient. A central processor 32 then retrieves the patient identification and communicates it to the sampling processor and printer, which prints the patient identification on the label 16 in a machine-readable format.

An automated analysis system 40 receives the container and appropriately positions it with appropriate analysis electronics and other equipment 42 as may be appropriate to perform the requested analysis. The analysis system 40 further includes a reader 44 which reads the label 16. A processor 46 receives the label information from the reader 44 and the analysis results from the automated analysis equipment 42 and communicates the information to the central hospital memory 30 for storage in the patient's records. If the label 16 on the container carries a unique serial number, the processor 32 at the central memory correlates the serial number with the serial number communicated from the sampling processor 28 at a sampling station. The central processor 32 or the analyzer processor 42 further correlates the biometric scanner information with a specific patient such that the analysis results are loaded into the proper patient record in the central patient memory 30. If the label carries an identification of the patient, then the processor loads the analysis results into the file of the patient identified on the label.

With reference to FIG. 2, a container 12′ is connected with a finger receiving structure 50 which carries the sensor surface 20, in this embodiment, a layer 52 of sticky foil or other material which is permanently deformed to hold a patient's fingerprint or other biometric when the finger or other body part is pressed against it. In the illustrated embodiment, the container is a capillary tube which is connected with a finger-shaped plastic surface that holds the sticky foil. The sticky foil is arranged relative to a needle end of the capillary tube such that pressing the patient's finger against the capillary tube needle requires the finger to be pressed against the sticky foil surface. In this manner, taking the blood or other biological sample, results in creating a mechanical record of a patient biometric indicator. Of course, other containers are contemplated, such as urine tubes, fecal matter sample containers, and the like. With a urine tube, for example, the sticky foil is adhered to a side surface of the urine tube. The patient presses their thumbprint into the sticky foil in conjunction with the sample collection. Other alternatives include a sticky foil tag that is attached to the container with a tether, a sticky foil surface on a removable cap or cover, and the like. Other medium which record a machine readable copy of fingerprints are also contemplated.

With reference to FIG. 3, when the container includes sticky foil or another machine readable means for holding or recording a biometric identification, the analysis system 40′ includes the sample analysis electronics and equipment 42′ as well as a biometric reader 54. In the illustrated fingerprint embossing embodiment of FIG. 2, the reader 54 includes a scanner which scans the sticky foil to analyze and read the patient's fingerprint. A processor 46′ combines the fingerprint analysis information with the analytical results and forwards them to the central hospital patient memory 30.

With reference to FIG. 4, each patient record 60 of the patient records memory 30 includes a patient identification 62 such as an identification number or name. It also includes an electronic representation 64 of biometric information, such as fingerprints. Further, the record includes an electronic representation 66 of DNA information. The DNA information may relate to a diagnosed illness and includes a preselected set of DNA markers. The DNA markers are selected for their ability to differentiate between patients and for their simplicity of determination during automated analysis. Analysis for various conditions often involves measuring 50-100 DNA markers. For breast cancer, typically about 80 markers are analyzed. However, for differentiating among patients, a much smaller number of markers may be adequate. For example, 10-12 markers, if properly chosen, could differentiate a patient from over 99% of the other patients in the hospital. Finally, a patient's medical record includes a medical history portion 68 which includes not only a history of previously diagnosed medical conditions and treatments, but also a history of test results, diagnostic images, and the like. The analysis system 40″ as a part of the sample analyzer 42′, includes a DNA analyzer 70. The DNA analyzer makes various DNA analyses as may be appropriate to the requested analysis. Further, if the DNA analysis does not call for the analysis of the selected DNA markers, the DNA analyzer further performs these preselected analyses. Where appropriate, the DNA analysis may be expedited using polymer chain reaction (PCR) technology.

The analysis system generates an analysis results record 72, which includes identification 74 of the sample analyzed. The identification can include the unique sample serial number read by the reader 44 from the analyzed container, an electronic representation of a fingerprint or other biometric measurement as measured by the scanner 54, a manually entered identification of the sample or the patient, or the like. The sample record further includes an electronic representation 76 of the analysis of the preselected DNA markers, as well as the other results 78 of the analysis performed.

When the analysis record 72 is sent to the central patient records memory 30, the central processor 32 looks at information previously entered into the patient memory to correlate the sample identification 74 with a patient identification 62. Once the corresponding patient has been identified, the processor 32 compares the preselected DNA markers 66 of the patient record with the DNA markers 76 of the analysis record. If the DNA markers match, then the analysis results are entered into the patient's records. If the DNA information does not match, then a warning is sent indicating that samples may have been inadvertently interchanged. Preferably, the central processor 32 further uses the DNA information 76 from the sample record to identify the patient to whom the test results belong. Although a comparison with all patient records can be performed, the search can often be simplified through the use of a priori information. For example, samples with a detected DNA mismatch can be held in a buffer and compared first with each other. As another example, where appropriate, the search may be limited to patients who are currently in the medical facility. Preferably, the central processor 32 generates a report identifying the most probable patient(s) to whom the analysis belongs. Alternately, the patients who have samples in testing can be identified in the patient database and the central processor 32 can look to match the patients with samples being analyzed to the analysis results. Of course, if a patient and test results cannot be paired up to a certainty, new samples are taken and the analysis is repeated.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A point of care diagnostics system comprising:
a container which receives a biological sample from a patient; and
an analyzer assembly which receives the biological container and generates (1) analysis results of the biological sample and (2) a biometrically based patient indicator.

2. The point of care diagnostics system according to claim 1, further including:
a biometric indicator means for generating a biometric indicator identifying the patient corresponding to the biological sample contemporaneously with receipt of biological sample in the container.

3. The point of care diagnostics system according to claim 2, wherein the biometric indicator means includes a fingerprint scanner (24) which scans a portion of the patient contemporaneously with receipt of the biological sample in the container.

4. The point of care diagnostics system according to claim 2, wherein the biometric indicator means includes a layer that makes a machine readable record of a patient's fingerprint when handling the container.

5. The point of care diagnostics system according to claim 4, wherein the analysis assembly includes a fingerprint reader for reading the patient's fingerprint from the layer, the patient indicator including an output from the fingerprint reader.

6. The point of care diagnostics system according to claim 2, wherein the biological sample is peripheral blood from a fingertip and the container includes a sampling needle which extends through a fingerprint recording means.

7. The point of care diagnostics system according to claim 1, further including:
a scanner for electronically reading a biometric measurement of the patient as the biological sample is collected;
a reader for reading an identification of the container as the sample is taken; and,
a sampling processor for communicating the biometric measurement and the container identification to a memory.

8. The point of care diagnostics system according to claim 7, wherein the analysis assembly includes:
a container identification reader; and,
an analyzer processor which communicates the patient identification with the analysis results to the memory.

9. The point of care diagnostics system according to claim 8, further including:
a central processor which receives the biometric information and the container information from the sampling processor and the analysis results and the container identification from the analyzer processor and correlates the analysis results with the patient from whom the biological sample was received.

10. The point of care diagnostics system according to claim 1, wherein:
the analysis system includes a DNA analyzer which analyzes the biological sample for a preselected set of DNA characteristics, the analysis system communicating the measured DNA characteristics to a central processor; and,
a plurality of patient records, each patient record including the preselected DNA characteristics of the patient, the central processor comparing the preselected DNA characteristics from the analyzer with patient record DNA characteristics to confirm that the test results are attributed to the correct patient.

11. The system according to claim 1, wherein the biometrically based patient indicator is based on the results of a preselected set of DNA markers and the analyzer system includes a DNA analyzer that analyzes the biological sample for the preselected set of DNA markers.

12. A method of point of care diagnosing comprising:
taking a biological sample of a patient;
analyzing the biological sample and generating analysis result information and a biometrically based patient identification for use in correlating the analysis results with the patient from whom the biological sample was taken.

13. The method according to claim 12, wherein:
the taking of the biological sample includes contemporaneously recording the patient's fingerprint.

14. The method according to claim 14, wherein recording the patient's fingerprint includes:
machine-readably marking a layer with the patient's fingerprint contemporaneously with the sample being collected; and
generating the patient identification includes machine reading the fingerprint layer and generating an electronic fingerprint identification signal.

15. The method according to claim 13, further including:
electronically scanning the patient's fingerprint and electronically reading an identification label on the container contemporaneously with taking the biological sample;
generating a signal indicative of the scanned fingerprint and the corresponding container identification;
contemporaneous with analyzing the biological sample, reading the container identification label;
with one or more processors:
correlating the container identification with the analysis results; and
correlating the analysis results to the scanned fingerprint.

16. The method according to claim 12, further including a patient database in which patient records are stored, each patient record including a patient identification, medical information, at least one of a biometric identifier of the patient, and a preselected subset of DNA characteristics of the patient, and wherein the method further includes:
correlating an electrical analysis results signal with a corresponding patient record.

17. The method according to claim 16, wherein correlating the electrical analysis results with a corresponding patient includes correlating a biometrically based patient identification generated contemporaneously with taking the sample with the biometric identifier of the corresponding patient record, and further including verifying correspondence between the corresponding patient and the sample by:
analyzing the biological sample for the preselected subset of DNA characteristics; and,
comparing the analyzed sample DNA characteristics with the DNA characteristics of the corresponding patient record.

18. The method according to claim 16, wherein the analyzing includes determining the preselected set of DNA characteristics from the biological sample, and wherein the correlating step includes matching the determined DNA characteristics with the DNA characteristics of the patient records.

19. The method according to claim 12, wherein the analyzing includes determining the preselected set of biometric characteristics from the biological sample, and wherein the correlating step includes matching the measured biometric characteristics with the biometric characteristics of the patient records.

20. An apparatus for performing the method of claim 13.
